(19) United States
(12) Patent Application Publication
(10) Pub. No.: US 2021/0307647 A1
(43) Pub. Date: Oct. 7, 2021

(54) APPARATUS, SYSTEM AND METHOD FOR MEASUREMENT OF A PATIENT BODY AND PORTIONS THEREOF

(71) Applicant: QUANTUM LABS, LLC, Union City, NJ (US)

(72) Inventors: Peter KAHN, New Haven, CT (US); Robert FOGERTY, Guilford, CT (US); Todd HIGGINS, Newtown, CT (US)

(73) Assignee: QUANTUM LABS, LLC, Union City, NJ (US)

(21) Appl. No.: 17/157,543

(22) Filed: Jan. 25, 2021

Related U.S. Application Data
(60) Provisional application No. 63/005,954, filed on Apr. 6, 2020.

(51) Int. Cl. A61B 5/107; A61B 5/00

(52) U.S. Cl. CPC A61B 5/1072; A61G 1/04; A61B 2505/01; A61B 5/6892

(57) ABSTRACT

A measuring apparatus for measurement of at least a portion of a patient body includes a measuring aid including a set of evenly spaced parallel lines that are divided at an intermediate parallel line into at least two sections in a direction perpendicular to the intermediate parallel line. The intermediate parallel line is a zero scale line, and each of the at least two sections is provided with individual scales of corresponding measurement units ascending from the intermediate parallel line for separately measuring discretely particular portions of the patient body. The measuring apparatus further includes a fixed medium on which the measuring aid is imprinted. The evenly spaced parallel lines are parallel in a transverse direction of the fixed medium, and the intermediate parallel line is approximately located at a middle point of the fixed medium in a longitudinal direction of the fixed medium.

### CROSS-REFERENCE TO RELATED APPLICATIONS

The present application claims priority to co-pending provisional application Ser. No. 63/005,954 filed Apr. 6, 2020, the contents of which are incorporated herein by reference.

### FIELD OF THE INVENTION

The present disclosure relates generally to the standardized measurement of a patient body in a hospital or clinical setting, and more particularly, to an apparatus, system and method for accurate, precise and uniform measurement of a patient body and portions thereof by use of a measuring aid imprinted upon a fixed medium, such as a hospital bedsheet, as more fully detailed and defined below.

### BACKGROUND

Hospitals and other facilities in the medical field currently lack a uniform system of measurement for a patient's body and/or portions thereof. As used herein, a patient may refer to a human being and/or an animal. Although this disclosure uses a human being patient in examples, this disclosure can be equally applicable to an animal patient. The effects of lacking a uniform system of measurement are \vide-ranging, costly, and potentially dangerous. By way of example, high risk and expensive medications such as epinephrine (a life-support infusion), rituximab (an anti-cancer drug), liposomal daunorubicine/cytarabine (an anti-cancer drug), and vancomycin (an antibiotic used in the treatment of serious drug-resistant infections) are dosed partially based on a patient's height. Miscalculations of a patient's height can therefore have serious implications. If there is an overmeasurement of patient height, a dose may be too high, creating an increased risk of medication toxicity and waste due to medication overuse. If there is an undermeasurement of patient height, a dose may be too low, producing an increased likelihood of treatment failure, prolonged illness, and potential waste due to unnecessarily prolonged treatments.

Patient height also factors into numerous additional clinical and treatment applications. By way of example, the National Kidney Foundation's recommended calculation for kidney function (which is itself used in dosing decisions), involves a mathematical equation in which patient height is a variable. Further, it is also significant that chemotherapy drugs and immunotherapy courses are also dosed in part based on patient height. Separately, ventilator settings for mechanical ventilation are based on patient height. And hospital nutrition decisions are typically made based on body mass index, which is itself adjusted for patient height.

In the Intensive Care Unit in particular, one of the standards of care is to provide mechanical ventilation through a ventilator, using an amount of air that is calibrated essentially to a value that is heavily dependent on patient height. In the event that an incorrect patient height is entered in the electronic medical record, that erroneous value may propagate throughout the patient's entire hospital course and may result in a number of significant quality and safety issues.

Despite these factors and other potential considerations, hospitals and other care providers do not have a standard, clinically valid, Health Insurance Portability and Accountability Act (HIPAA)-compliant system for patient height measurement that can be used for most or all patients. Indeed, smartphone apps used for patient height measurement are typically not HIPAA-compliant and may not be validated for clinical use. Additionally, handheld rulers or measuring tapes are not always accurate for bedbound patients. Standing measurement devices are also not practical for the significant population (if not majority) of hospitalized patients who cannot be mobilized without assistance. Practitioner estimates (e.g., visual estimation or asking a family member) and self-reports of height are also often inaccurate. Accordingly, there exists a need for a practical, precise, accurate, and uniform system of patient height measurement in the medical field.

Prior art exists with respect to the measurement of a human body using a bedding article, see, e.g., Measuring Bedding Articles and Methods for Measuring Using Same, U.S. Pat. No. 7,340,842, but the existing system of measurement is inexact, not designed to guarantee precise measurement of the human body, and requires the individual to be positioned in a particular location. The existing art is also not designed for use in clinical or medical applications, including in environments where HIPAA compliance is required, or capable of being readily deployed in particular embodiments suitable for the medical context, such as with a fitted sheet for a hospital bed.

Prior art also exists in devices and methods designed to determine particular information relating to user physique for particular discrete purposes. See, e.g., Pillow Selection and Sleeper Appraisal, U.S. Pat. No. 8,033,030 (neck measuring device for pillow selection); System for Determining Optimal Mattress Characteristics for Individuals, U.S. Pat. No. 9,456,703 (system for determining optimal mattress type for an individual based on measurement of body), Such art, however, does not offer a general system of measurement that is suitable to a variety of applications and implementations in the medical field, and is rather geared towards measurement or calculation of solely one output for one explicit purpose. Moreover, some such art is costly, technically complex, and not well suited to widespread adoption in a variety of settings.

As discussed below, the present disclosure advances the art, by providing a uniform, precise means for measurement of portions of and/or the entirety of a patient body in the clinical environment, for use in diagnostic and other treatment applications.

### SUMMARY

Embodiments of the present disclosure provide a measuring apparatus for measurement of at least a portion of a patient body. The measuring apparatus comprises a measuring aid including a set of evenly spaced parallel lines. A distance between each pair of adjacent parallel lines is configured to tabulate a total length of the patient body or a portion thereof being measured. The set of evenly spaced parallel lines are divided at an intermediate parallel line into at least two sections in a direction perpendicular to the intermediate parallel line, the intermediate parallel line being a zero scale line of the set of evenly spaced parallel lines, and each of the at least two sections is provided with individual scales of corresponding measurement units ascending from the intermediate parallel line for separately measuring discretely particular portions of the patient body. The measuring apparatus further comprises a fixed medium having a first surface and a second surface opposing the first surface. The measuring aid is imprinted on at least one of the first surface and the second surface, the set of evenly spaced parallel lines are parallel in a transverse direction of the fixed medium, and the intermediate parallel line is approximately located at a middle point of the fixed medium in a longitudinal direction of the fixed medium.

Embodiments of the present disclosure provide a measuring system for accurate, precise and uniform measurement of at least a portion of a patient body without moving the patient body. The measuring system comprises a fixed medium having a first surface and a second surface opposing the first surface, and a measuring aid imprinted on at least one of the first surface and the second surface. The measuring aid includes a set of evenly spaced parallel lines, a distance between each pair of adjacent parallel lines is configured to tabulate a total length of the portion of the patient body or a component thereof being measured. The set of evenly spaced parallel lines are divided at an intermediate parallel line into at least two sections in a direction perpendicular to the intermediate parallel line, the intermediate parallel line being a zero scale line of the set of evenly spaced parallel lines, each of the at least two sections is provided with individual scales of corresponding measurement unit for separately measuring discretely particular portions of the patient body, the set of evenly spaced parallel lines are parallel in a transverse direction of the fixed medium, and the at least two sections are configured to have approximately equal number of parallel lines on either side of the intermediate parallel line.

Embodiments of the present disclosure provides a method of performing an accurate measurement of at least a portion of a patient body without moving the patient body. The method comprises: having the patient body laid over a measuring aid imprinted on a fixed medium, wherein the fixed medium is configured to have a first surface and a second surface opposing the first surface, the measuring aid is imprinted on at least one of the first surface and the second surface, the measuring aid includes a set of evenly spaced parallel lines, a distance between each pair of adjacent parallel lines is configured to tabulate a total length of the portion of the patient body or a component thereof being measured. The set of evenly spaced parallel lines are divided at an intermediate parallel line into at least two sections in a direction perpendicular to the intermediate parallel line, the intermediate parallel line being a zero scale line of the set of evenly spaced parallel lines, each of the at least two sections is provided with individual scales of corresponding measurement unit for separately measuring discretely particular portions of the patient body, the set of evenly spaced parallel lines are parallel in a transverse direction of the fixed medium, and the intermediate parallel line is approximately located at a middle point of the fixed medium in a longitudinal direction of the fixed medium; measuring a top half of the patient body by counting the number of parallel lines from the intermediate parallel line in a corresponding section of the at least two sections that is laid over by the top half of the patient body; multiplying the number of parallel lines laid over by the top half of the patient body with a first corresponding measurement unit to obtain a length of the top half of the patient body; measuring a lower half of the patient body by counting the number of parallel lines from the intermediate parallel line in another section of the at least two sections that is laid over by the lower half of the patient body; multiplying the number of parallel lines laid over by the lower half of the patient body with a second corresponding measurement unit to obtain a length of the lower half of the patient body; and summing the length of the lower half of the patient body and the length of the top half of the patient body to obtain a total length of the patient body.

Further features of the disclosed apparatus, method and system, and the advantages offered thereby, are explained in greater detail hereinafter with reference to specific example embodiments illustrated in the accompanying drawings.

### BRIEF DESCRIPTION OF THE DRAWINGS

FIG. 1 illustrates an example measurement apparatus comprising a measuring aid and a fixed medium on which the measuring aid is imprinted, according to embodiments of the present disclosure.

FIG. 2 illustrates an example measurement apparatus comprising a measuring aid and a bedsheet on which the measuring aid is imprinted, which may be used in a hospital room setting, according to embodiments of the present disclosure.

FIG. 3 illustrates an example measurement apparatus comprising a measuring aid and a stretcher on which the measuring aid is imprinted, according to embodiments of the present disclosure.

FIG. 4 illustrates an example measurement apparatus comprising a measuring aid and a backboard on which the measuring aid is imprinted, according to embodiments of the present disclosure.

FIG. 5A illustrates an example measurement apparatus comprising a measuring aid and a fixed medium including a first surface on which the measuring aid is imprinted, according to embodiments of the present disclosure.

FIG. 5B illustrates the example measurement apparatus in FIG. 5A, in which the fixed medium includes a second surface on which a fastener mechanism is provided for securing the measurement apparatus to a patient support, according to embodiments of the present disclosure.

FIG. 6 illustrates an example measurement apparatus laid over a medical exam table, according to embodiments of the present disclosure.

FIG. 7 illustrates an example measurement apparatus comprising a measuring aid having a subset of smaller scale measuring units and a fixed medium on which the measuring aid is imprinted, according to embodiments of the present disclosure.

FIG. 8 illustrates an example measurement apparatus comprising a measuring aid including a subset of additional measuring aids and a fixed medium on which the measuring aid is imprinted, according to embodiments of the present disclosure.

FIG. 9 illustrates a flow chart for an example measurement method, according to embodiments of the present disclosure.

### DETAILED DESCRIPTION OF THE EMBODIMENTS

The following description is intended to convey a thorough understanding of the embodiments described by providing a number of specific example embodiments and details. It should be appreciated, however, that the present disclosure is not limited to these specific embodiments and details, which are examples only. It is further understood that one of ordinary skill in the art, in light of known apparatuses, systems and methods, would appreciate the use of the invention for its intended purposes and benefits in any number of alternative embodiments, depending on specific design and other needs.

The present disclosure is not to be limited in terms of the particular embodiments described herein, which are intended as illustrations of various aspects. Many modifications and variations can be made without departing from its spirit and scope, as may be apparent. Functionally equivalent systems, methods and apparatuses within the scope of the present disclosure, in addition to those enumerated herein, may be apparent from the below representative descriptions. Such modifications and variations are intended to fall within the scope of the appended representative claims. The present disclosure is to be limited only by the terms of the appended representative claims, along with the full scope of equivalents to which such representative claims are entitled. It is also to be understood that the terminology used herein is for the purpose of describing particular embodiments only, and is not intended to be limiting.

The below description, along with its associated embodiments, has been presented for purposes of illustration only. It is not exhaustive and does not limit the invention to the precise form disclosed. Those skilled in the art may appreciate from the below description that modifications and variations are possible in light of the below teachings or may be acquired from practicing the disclosed embodiments. For example, the steps described need not be performed in the same sequence discussed or with the same degree of separation. Likewise various steps may be omitted, repeated, or combined, as necessary, to achieve the same or similar objectives. Accordingly, the invention is not limited to the below-described embodiments, but instead is defined by the appended claims in light of their full scope of equivalents.

In the present disclosure, various preferred embodiments have been described with references to the accompanying drawings. It may, however, be evident that various modifications and changes may be made thereto, and additional embodiments may be implemented, without departing from the broader scope of the invention as set forth in the claims that follow. The present disclosure and drawings are accordingly to be regarded in an illustrative rather than restrictive sense.

As discussed above, conventional measuring devices which have been suggested as uniform standards of measure have significant drawbacks which undermine their viability. For example, measuring applications and similar technologies are widely available on many smartphones and other devices. For a number of reasons, however, these technologies oftentimes present potential information technology (IT) vulnerabilities or compliance risks (e.g., unintentional or intentional retention of total-body scans or other data which may constitute private health information (PHI), and potential security lapses with respect to this information). In contrast, the measuring apparatuses, systems and methods disclosed herein do not present a compliance threat or other risk, while providing for accurate and uniform measurement of patients and other subjects.

Various embodiments of the present disclosure provide apparatuses, systems and methods for accurate, precise and uniform measurement of a patient body and portions thereof by use of a measuring aid imprinted upon a fixed medium, for example, a hospital bedsheet. The disclosed apparatuses, systems and methods are capable of conveying an accurate, precise and uniform measurement of the patient body and portions thereof, regardless of whether the patient is centered (longitudinally or transversely) on a hospital bed or whether the patient is more on one side than the other side on the hospital bed, which can provide highly accurate height and length measurements of the patient in healthcare and other potential scenarios in which a height or length measurement is important for functioning. For example, by having a zero scale line positioned at or near the middle point of the bedsheet where the middle point is a point dividing the bedsheet into two substantially similar sized sections along a longitudinal direction of the bedsheet and the zero scale line runs across the bedsheet in a transverse direction of the bedsheet, the measurements can run all the way across the bedsheet obviating concerns about the position of the patient body.

The measuring aid may include a set of evenly spaced parallel lines imprinted on the fixed medium. The distance between the parallel lines can be used to tabulate a total length of a patient body or a component thereof being measured. The set of parallel lines of the measuring aid may be divided into sections or sets with individual scales of desired units of measure, which can be used separately to measure discrete and particular portions of a patient body, or summed or otherwise computed mathematically to reach an aggregate measurement of a desired part of the patient body, up to and including the whole patient body. For example, the set of parallel lines may be divided into two sections by an intermediate parallel line having a zero scale, a first section of which includes parallel lines on a first side of the intermediate parallel line and a second section of which includes parallel lines on a second side of the intermediate line. The parallel lines in the first section and the parallel lines in the second section may be provided with individual scales of corresponding measurement units ascending from the intermediate parallel line for separately measuring discretely particular portions of the patient body. The use of the measuring aid disclosed herein can provide an accurate and standardized measurement of a patient body in subjects with all form of abilities and conditions.

In particular, the measuring aid as disclosed herein can provide a novel solution to measuring height or length for patients that are immobile, as well as other vital statistics regardless of patient mobility. Being able to measure a patient without moving their body can be particularly important in a critical care environment because, for example, moving a patient who is attached to life support apparatus such as a ventilator or continuous hemodialysis can be extremely dangerous.

As discussed herein, the disclosed apparatuses, systems and methods may be deployed across various embodiments in order to suit different applications, including but not limited to, in the clinical and emergency medicine and emergency intervention settings, offering a standardized system of height or length measurement that is, among other things, fully HIPAA compliant. For example, the apparatuses, systems and methods disclosed herein do not record total-body images or present compliance risks and IT vulnerabilities.

The present disclosure therefore can offer a nonobvious solution for standardized, clinical height or length measurement of a full patient body through the measuring aid, which may be divided into a particular number of sections or sets for precise measurement of individual portions of the patient body as well as the body as a whole through the measuring system disclosed herein.

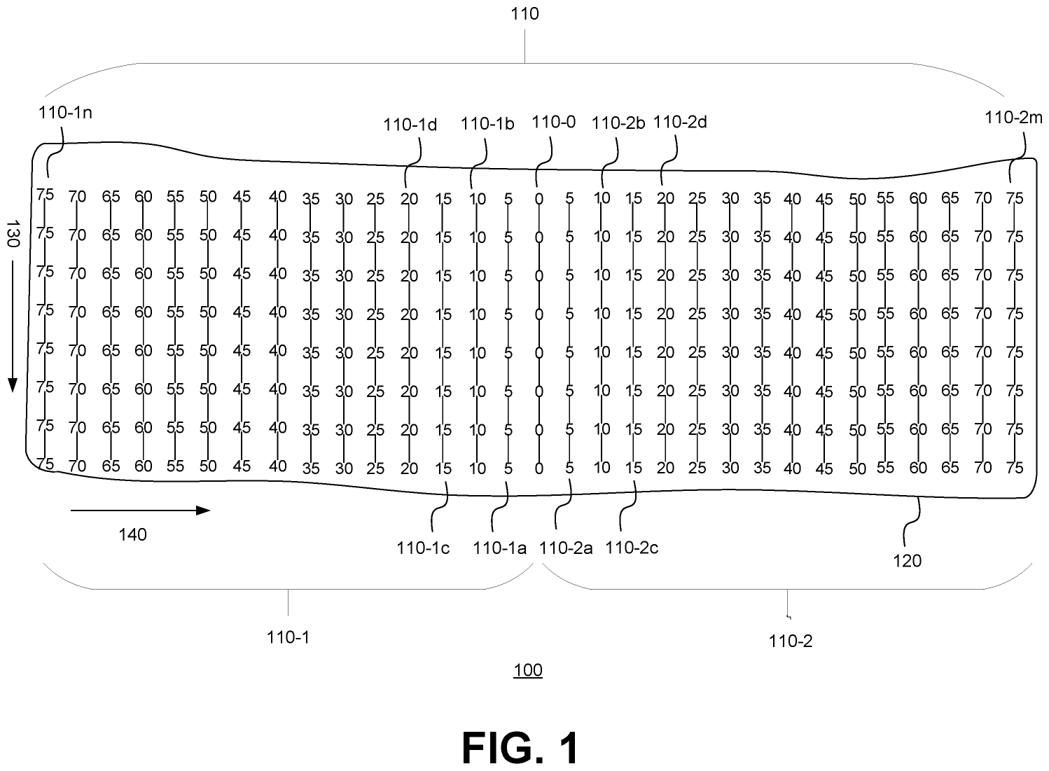

FIG. 1 illustrates an example measurement apparatus 100 comprising a measuring aid and a fixed medium on which the measuring aid is imprinted, according to embodiments of the present disclosure. The measurement apparatus 100 may include a measuring aid 110 comprising a set of evenly spaced parallel lines (e.g., 110-1a, 110-1b . . . 110-1n, an intermediate line 110-0 and 110-2a, 110-2b . . . 110-2m, where n and m are positive integers and n and m may be the same number or different numbers). The measurement apparatus 100 may further include a fixed medium 120 on which the measuring aid 110 is imprinted.

The measuring aid 110 may be divided into sections or sets with individual scales of the implemented units of measure, which can be used separately to measure discretely and particular portions of a patient body. As shown in FIG. 1, the set of evenly spaced parallel lines of the measuring aid 110 are divided at the intermediate parallel line 110-0 into at least two sections in a direction perpendicular to the intermediate parallel line 110-0. A first section of the at least two sections is designated as 110-1 in FIG. 1 and a second section of the at least two sections is designated as 110-2 in FIG. 1. The intermediate parallel line 110-0 is a zero scale line (e.g., zero inch) of the set of evenly spaced parallel lines. The direction perpendicular to the intermediate parallel line 110-0 may be indicated by the arrow 140 in FIG. 1.

Each of the at least two sections (the first section 110-1 and the second section 110-2) may be provided with individual scales of corresponding measurement units ascending from the intermediate parallel line 110-0 for separately measuring discretely particular portions of the patient body. The corresponding measurement unit may be a metric measurement system unit (e.g., centimeter and meter), an English measurement system unit (e.g., inch and foot), or a combination thereof. For example, the first section 110-1 and the second section 110-2 may both employ the metric measurement system unit or the English measurement system unit. Alternatively, the first section 110-1 may employ the metric measurement system unit while the second section 110-2 may employ the English measurement system unit.

As illustrated in FIG. 1, each of the evenly spaced parallel lines of the measuring aid 110 may be labeled with one or more number of a same value indicative of a corresponding scale or distance measured in the corresponding measurement unit from the intermediate parallel line 110-0. For example, the set of zeros embedded in the intermediate parallel line 110-0 indicate that the intermediate parallel line 110-0 is at zero inch or centimeter, the set of fives embedded in each of the parallel line 110-1a and the parallel line 110-2a indicate that the parallel line 110-1a and the parallel line 110-2a each is distanced five inches or centimeters from the intermediate parallel line 110-0, and so forth. The numbers may also be printed in different orientations so that their value can be read from either side of the measuring aid 110. For example, some of the fives in parallel line 110-1a could be printed upright while others are printed upside-down thus allowing viewers on opposite sides of the measuring aid 110 to view numbers that appear upright from their position.

The measuring aid 110 can be imprinted on the fixed medium 120 using any suitable method or technique. For example, the measuring aid 110 may be screen printed, inkjet printed, laser printed, or thermal printed on the fixed medium 120.

The fixed medium 120 may have a first surface and a second surface opposing the first surface, for example, a front surface and a back surface. The measuring aid 110 is imprinted on at least one of the first surface and the second surface. That is, the measuring aid 110 may be imprinted on either of the first surface and the second surface, or may be imprinted on both the first surface and the second surface. The set of evenly spaced parallel lines of the measuring aid 110 may imprinted parallel in a transverse direction of the fixed medium 120 (the transverse direction being illustrated by the arrow 130 in FIG. 1). The intermediate parallel line 110-0 is approximately located at a middle point of the fixed medium 120 in a longitudinal direction of the fixed medium 120 (the longitudinal direction being illustrated by the arrow 140 in FIG. 1).

The fixed medium 120 may be soft, hard, flexible or rigid, and may be made of any suitable materials, such as cotton, fabric, plastic, wood, metal, paper, and so forth. The fixed medium 120 may embody any suitable form or shape, including, but not limited to, a bedsheet, a stretcher, a backboard, and a bench.

In some embodiments, the at least two sections may be configured to have approximately equal number of parallel lines on either side of the intermediate parallel line 110-0. For example, the number of the parallel lines in the section 110-1 may be twenty, and the number of the parallel lines in the section 110-2 may be nineteen, twenty or twenty one.

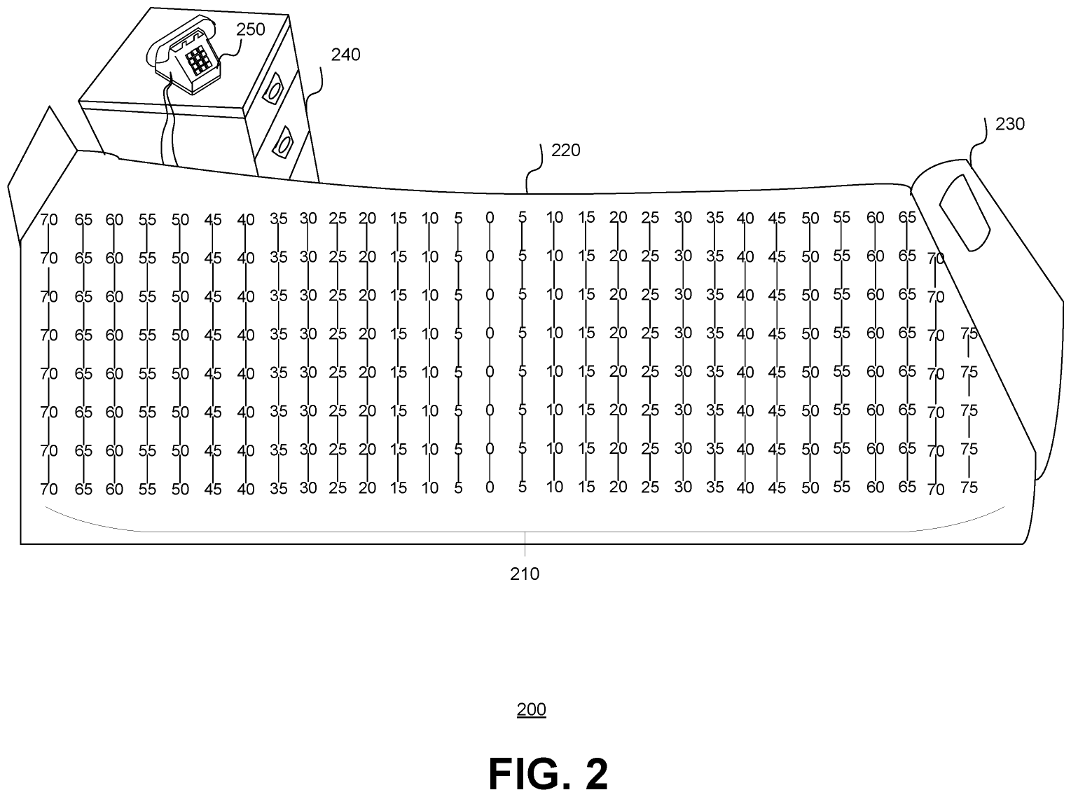

FIG. 2 illustrates an example measurement apparatus 200 comprising a measuring aid 210 and a bedsheet 220 on which the measuring aid 210 is imprinted, according to an embodiment of the present disclosure. In the measurement apparatus 200, the bedsheet 220 corresponds to the fixed medium 120 in FIG. 1, which can enable length or height measurements of a patient body while the patient is lying in bed over which the measurement apparatus 200 is covered.

The measurement apparatus 200 may be used for measuring length or height of a patient body or parts thereof in a hospital room environment, for example, in hospital units or hospital emergency departments. As shown in FIG. 2, the measurement apparatus 200 may be laid over a hospital bed 230. When a patient is lying on the measurement apparatus 200, the whole body of the patient or parts thereof can be accurately measured without bothering the patient (e.g., no need to ask the patient to move around on the bed).

As shown in FIG. 2, the measuring aid 210 includes two sets of evenly spaced, parallel lines, with a line “zero” cutting laterally through the true midpoint of the bedsheet 220, such that accurate and discrete measurement (in metric or imperial or both) can be made via the measurement apparatus 200 for the top half and the lower half of the patient body separately, which may then be added together to represent a patient's overall height or length. In the measurement apparatus 200, the spacing between the evenly spaced, parallel lines of the measuring aid 210 are separated in a discrete number of inches or centimeters or both so as to allow accurate measurements of a patient or subject's body.

Furthermore, in the measurement apparatus 200, the parallel lines extend in both the transverse direction and the longitudinal direction across the bedsheet 220 such that patient body extremities (e.g., a forearm) or medical conditions present on those extremities can be measured without moving the patient. The medical conditions present on those extremities may include, but not limited to, an infection around an IV site, a wound, or the size of a pressure ulcer. The extension of the parallel lines may also allow measurement of medical devices being used to treat the patient without moving the patient body. The medical devices may include, but not limited to, nasogastric tubes, bandages, and slings/support apparatus.

The measuring aid 210 may be imprinted only on one surface of the bedsheet 220. In some embodiments, the measuring aid 210 may be imprinted on both surfaces of the bedsheet 220 (e.g., a top surface and a bottom surface or a front surface and a back surface). Imprinting the measuring aid 210 on both surfaces of the bedsheet 220 may provide additional benefits. For example, when the measuring aid 210 on one surface of the bedsheet 220 fades such that measurements cannot be accurately made, the measuring aid 210 on another surface of the bedsheet 220 may be used to make the measurements instead of discarding the measurement apparatus 200.

Also shown in FIG. 2 are an example nightstand table 240 and a telephone 250 that are just for illustrative purpose indicating that the measurement apparatus 200 can be used in a hospital room setting.

Although the present invention has been described by way of example, it is to be noted here that various changes and modifications will be apparent to those skilled in the art. Therefore, unless such changes and modifications depart from the scope of the present invention, they should be construed as being included therein.

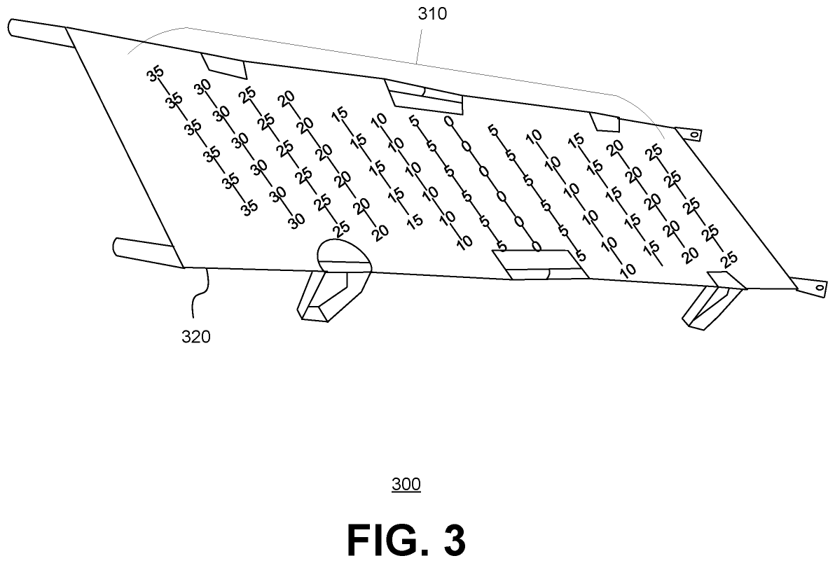

For instance, in one alternative embodiment of the present invention, the measuring aid may be deployed for use on stretchers conventionally found in ambulances. FIG. 3 illustrates a measurement apparatus 300 comprising a measuring aid 310 and a stretcher 320 on which the measuring aid 310 is imprinted, according to an example embodiment of the present disclosure. In the measurement apparatus 300, the fixed medium 120 in FIG. 1 embodies the stretcher 320.

The measurement apparatus 300 may be provided for use, for example, in emergency medicine situations. As shown in FIG. 3, the measuring aid 310 includes two sets of evenly spaced, parallel lines, with a line “zero” cutting laterally through the midpoint of the stretcher 320 or approximately the midpoint of the stretcher 320, such that accurate and discrete measurement (in metric or imperial or both) can be made via the measurement apparatus 300 for the top half and the lower half of a patient body separately, which may then be added together to represent a patient's overall height or length. In the measurement apparatus 300, the spacing between the evenly spaced, parallel lines of the measuring aid 310 are separated in a discrete number of inches or centimeters or both so as to allow accurate measurements of a patient or subject's body who is lying on the stretcher 320.

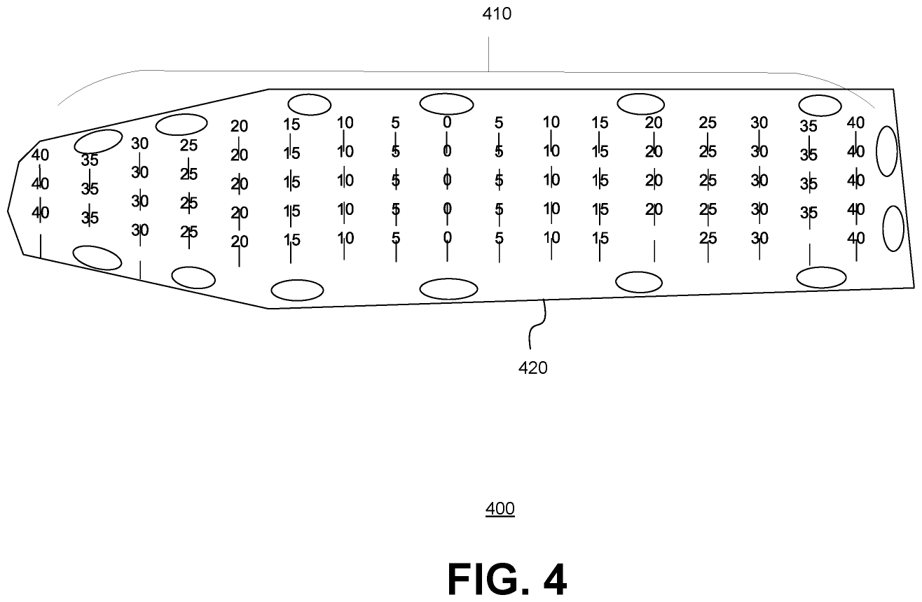

In another alternative embodiment, the measuring aid can be deployed for use on backboards typically involved in emergency medical treatment applications such as tending to immobilized persons suffering from neck injuries. FIG. 4 illustrates a measurement apparatus 400 comprising a measuring aid 410 and a backboard 420 on which the measuring aid 410 is imprinted, according to an example embodiment of the present disclosure. In the measurement apparatus 400, the fixed medium 120 in FIG. 1 embodies the backboard 420.

The measurement apparatus 400 may be provided for use, for example, in emergency medicine situations, such as for neck injuries. As shown in FIG. 4, the measuring aid 410 includes two sets of evenly spaced, parallel lines, with a line “zero” cutting laterally through the midpoint of the backboard 420 or approximately the midpoint of the backboard 420, such that accurate and discrete measurement (in metric or imperial units or both) can be made via the measurement apparatus 400 for the top half and the lower half of a patient body separately, which may then be added together to represent a patient's overall height or length. In the measurement apparatus 400, the spacing between the evenly spaced, parallel lines of the measuring aid 410 are separated in a discrete number of inches or centimeters or both so as to allow accurate measurements of a patient or subject's body who is lying on the backboard 420.

In some embodiments, a measuring aid may be imprinted on a first surface (e.g., a front surface) of a fixed medium while a second surface (e.g., a back surface) of the fixed medium may be provided with a fastener mechanism for facilitating the measurement apparatus to be attached to a suitable patient support or other equipment.

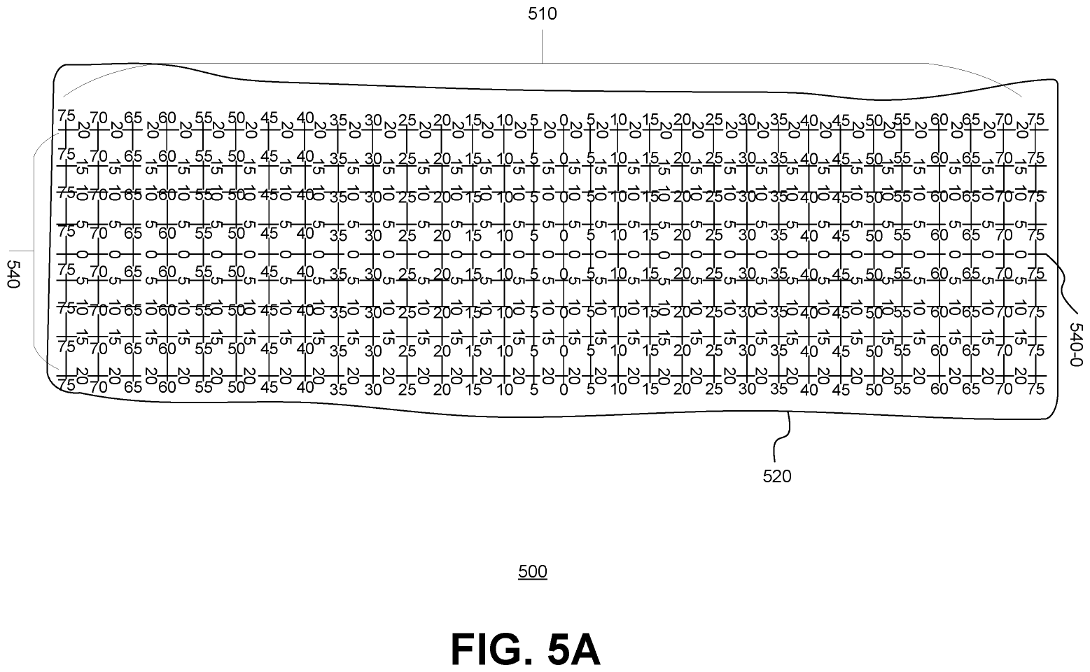

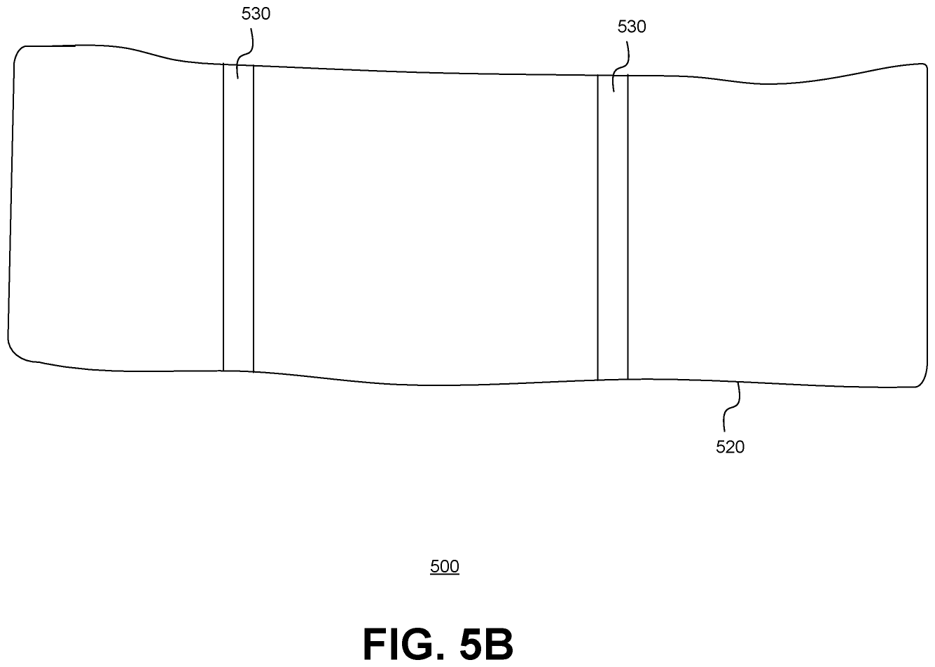

FIG. 5A illustrates an example measurement apparatus 500 comprising a measuring aid 510 and a fixed medium 520 including a first surface on which the measuring aid 510 is imprinted, according to embodiments of the present disclosure. FIG. 5B illustrates the example measurement apparatus 500, in which the fixed medium 520 includes a second surface on which a fastener mechanism 530 is provided for securing the measurement apparatus 500, either temporarily or permanently, to a patient support apparatus or other equipment, according to embodiments of the present disclosure.

In some embodiments, the measurement apparatus 500 may further comprise a second measuring aid 540. The second measuring aid 540 may include a set of evenly spaced parallel lines that form a certain angle with respect to the set of evenly spaced parallel lines of the measuring aid 510 (e.g., a 90 degree angle or perpendicular).

The measuring aid 540 may also be divided into sections or sets with individual scales of the implemented units of measure, which can be used separately to measure discretely and particular portions of a patient body. As shown in FIG. 5A, the set of evenly spaced parallel lines of the measuring aid 540 are divided at an intermediate parallel line 540-0 (zero scale) into at least two sections in a direction perpendicular to the intermediate parallel line 540-0. The intermediate parallel line 540-0 is a zero scale line (e.g., zero inch) of the set of evenly spaced parallel lines of the measuring aid 540. The direction perpendicular to the intermediate parallel line 540-0 may be the transverse direction of the fixed medium 520.

Each of the at least two sections of the measuring aid 540 may be provided with individual scales of corresponding measurement units ascending from the intermediate parallel line 540-0 for separately measuring discretely particular portions of the patient body. The corresponding measurement unit may be a metric measurement system unit (e.g., centimeter and meter), an English measurement system unit (e.g., inch and foot), or a combination thereof. For example, the first section and the second section of the measuring aid 540 may both employ the metric measurement system unit or the English measurement system unit. Alternatively, the first section of the measuring aid 540 may employ the metric measurement system unit while the second section of the measuring aid 540 may employ the English measurement system unit.

As illustrated in FIG. 5A, each of the evenly spaced parallel lines of the measuring aid 540 may also be labeled with one or more number of a same value indicative of a corresponding scale or distance measured in the corresponding measurement unit from the intermediate parallel line 540-0. For example, the set of zeros embedded in the intermediate parallel line 540-0 indicate that the intermediate parallel line 540-0 is at zero inch or centimeter, and so forth.

The fastener mechanism 530 may include, but is not limited to, at least one adhesive patch, at least one hook-and-loop fastener, or combination thereof. When the measurement apparatus 500 is applied to the patient support apparatus, the measuring aid 510 on the first surface of the fixed medium 520 may be orientated to face a patient for measuring the patient while the patient lying on the measurement apparatus 500, and the second surface of the fixed medium 520 is oriented to face a surface of the patient support apparatus and in contact with the surface of the patient support apparatus such that the fastener mechanism 530 can facilitate securing the measurement apparatus 500 to the patient support apparatus or other equipment. In other embodiments, the fastener mechanism 530 may be used to secure the measurement apparatus 500 to the patient. In such an embodiment, the fixed medium 520 may be a flexible, transparent or translucent film, such as an incise drape used in surgery. The measuring aid 510 may be printed on the fixed medium 520 to facilitate taking measurements during surgery or other procedures, such as the length of an incision or location of an item of interest on the patient body. For example, an incise drape imprinted with measuring aid may be secured to the patient body using an adhesive, which may also be impregnated with an antiseptic or antimicrobial agent such as iodophor. The measuring aid 510 may be imprinted on the surface of the fixed medium 520 oriented away from the patient or alternatively, imprinted on the surface facing the patient skin such that the markings of the measuring aid remain visible in the adhesive residue left on the patient skin when the fixed medium 520 is removed.

The patient support apparatus may include, but is not limited to, a bench, a doctor exam table, a stretcher, or a backboard. For example, instead of imprinting a measuring aid onto a stretcher (e.g., FIG. 3), the measurement apparatus 500 may be attached through the at least one fastener to a stretcher for use in emergency medicine situations, such that the fixed medium 520 is secured to the stretcher while a portion of the patient body is measured. Alternatively, instead of imprinting a measuring aid onto a backboard (e.g., FIG. 4), the measurement apparatus 500 may be attached through the at least one fastener to a backboard for use in emergency medicine situations, such that the fixed medium 520 is secured to the backboard while a portion of the patient body is measured.

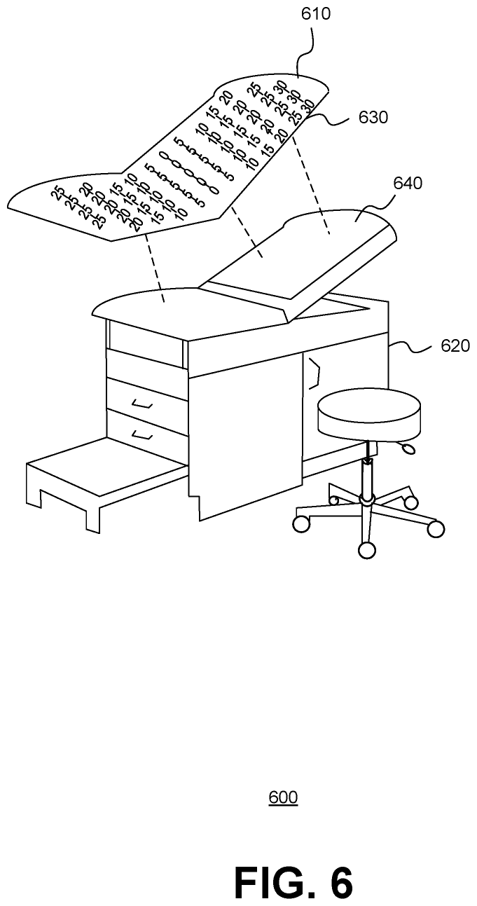

FIG. 6 illustrates an example usage of a measurement apparatus 610 that can be laid over a medical exam table 620 in a doctor exam room setting. The measurement apparatus 610 can have a fastener mechanism or may not have a fastener mechanism. The measuring aid may be imprinted on only one surface of the fixed medium of the measurement apparatus 600 or may be imprinted on both surfaces of the fixed medium of the measurement apparatus 600. The fixed medium of the measurement apparatus 600 may be made of flexible materials, such that the fixed medium is adapted to spread over the medical examination table 620. The second surface 630 of the fixed medium is in contact with a top surface 640 of the medical examination table 620 such that the portion of the patient body is measured using the measuring aid imprinted on the first surface of the fixed medium. Likewise, the measurement apparatus 610 can be spread over a backboard, a stretcher, or other patient support wherever suitable.

In some embodiments, doctors may use a roll of paper to cover their examination tables, as seen oftentimes in doctor's examination rooms. The roll of paper may be fixed to one end of the examination table. In such situations, the measuring aid may be repeatedly printed on the fixed medium (i.e., the roll of paper in this case), such that each measuring aid (e.g., in FIG. 6) may be approximately adapted to the dimension of the top surface 640 of the examination table 620. The doctor can tear off the section used by the prior patient and unroll a new section for the next patient. That is, the doctor can unroll a new “measuring aid” for each patient, which can effectively reduce or eliminate contamination risk and enhance hygiene.

In some embodiments, the evenly spaced parallel lines in at least one of the at least two sections of the measuring aid (e.g., the measuring aid 110) may be further divided into a subset of smaller scale evenly spaced parallel lines, which may offer more precise measurements of a patient body or parts thereof.

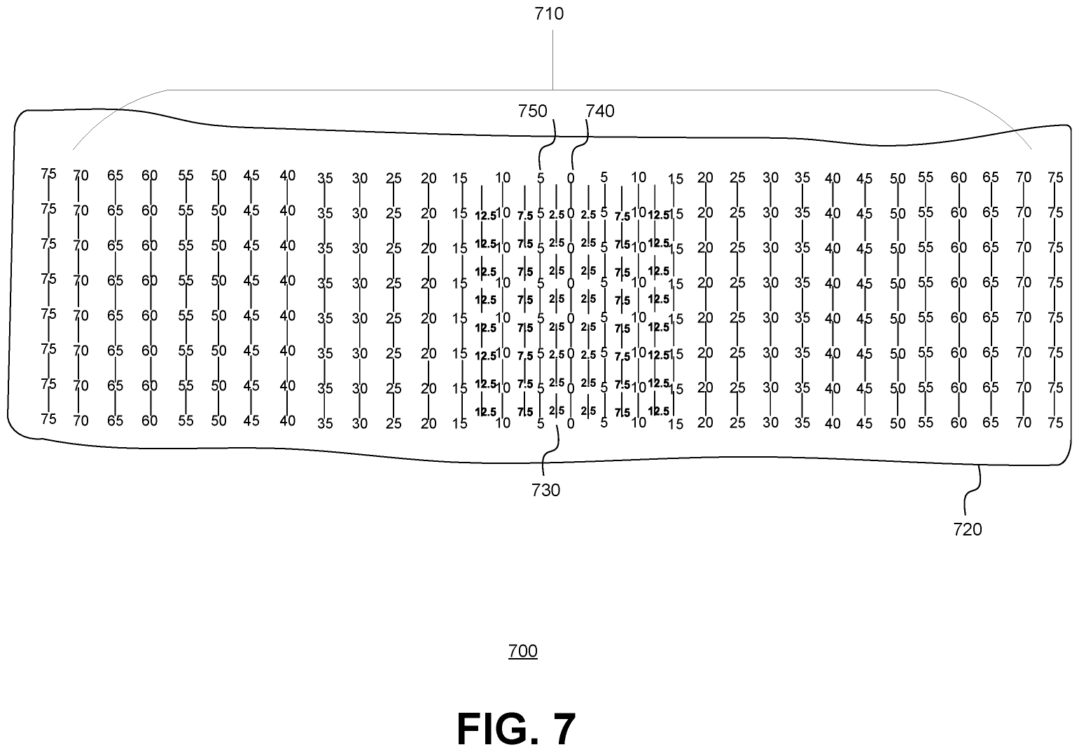

FIG. 7 illustrates a measurement apparatus 700 comprising a measuring aid 710 having a subset of smaller scale measuring units and a fixed medium 720 on which the measuring aid 710 is imprinted. The subset of smaller scale measuring units may correspond to a set of parallel lines each of which divides a corresponding large unit into two small equal scale units. For example, the parallel line 730 marked with 2.5 divides a large scale between the parallel line 740 marked with zero and the parallel line 750 marked with 5 into two equally small scales. Alternatively, each of the smaller scale measuring units may divide a corresponding large scale into two small unequal scales. The subset of smaller scale lines may or may not include numbers corresponding to the smaller scale.

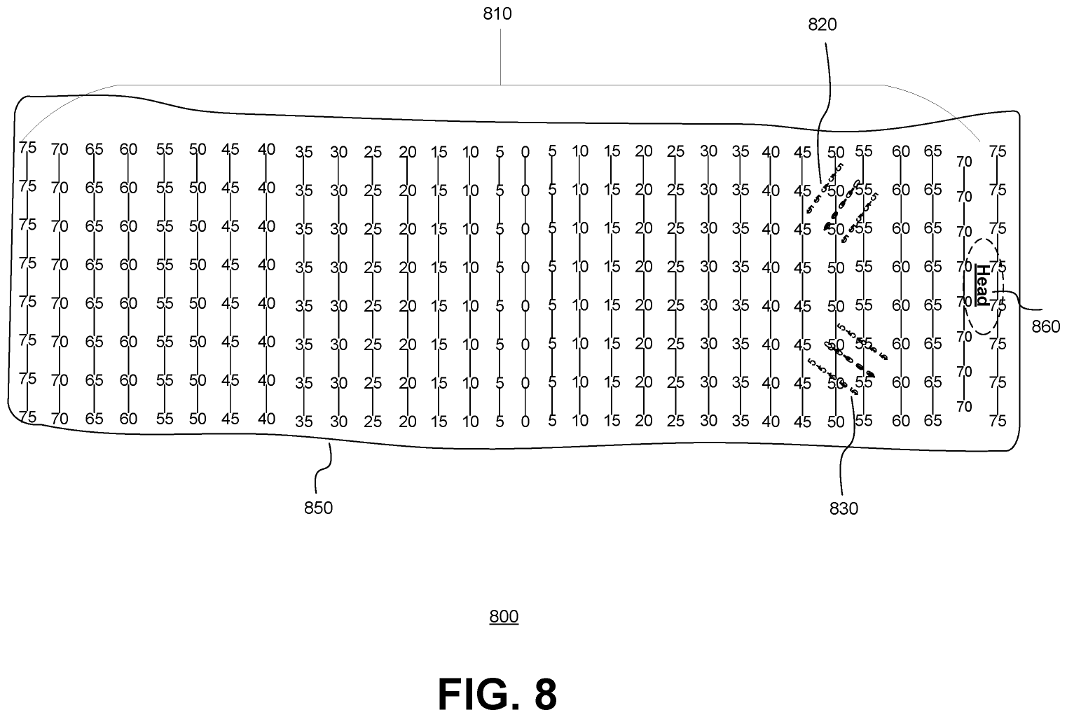

In some embodiments, a measurement apparatus may comprise a primary measuring aid and at least one secondary measuring aid for measuring a particular part of a patient body. FIG. 8 illustrates an example measurement apparatus 800 comprising a primary measuring aid 810 and at least one secondary measuring aid 820 and 830 according to embodiments of the present disclosure. The primary measuring aid 810 and the at least one secondary measuring aid 820 and 830 are imprinted on a fixed medium 850. The at least one second measuring aid 820 and 830 may each comprise a set of evenly spaced parallel lines in which an intermediate line is set to be a zero scale line. The at least one second measuring aid 820 and 830 may form an angle with respect to the primary measuring aid, for example, according to posture of a patient body. For example, in this example embodiment, when a patient is lying on the measurement apparatus 800 with face up and head at the position 860, the at least one second measuring aid 820 and 830 may be used to measure the right arm (or the right hand) and the left arm (or left hand), respectively.

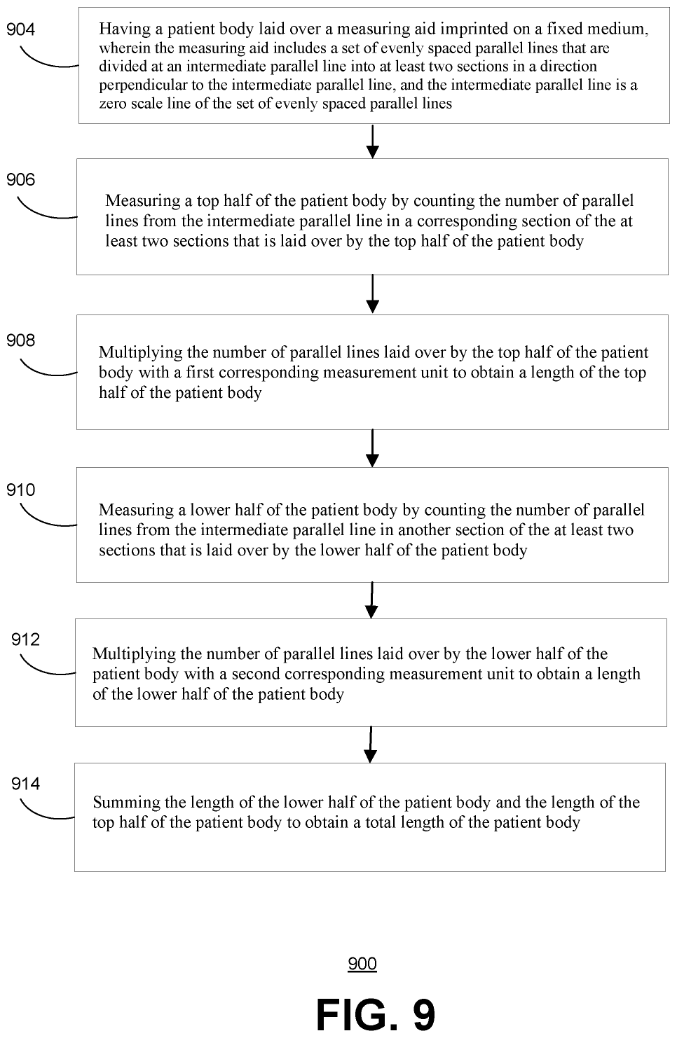

As described above, the measurement apparatus disclosed herein can be used to measure heights or lengths of patients. FIG. 9 illustrates a flow chart for an example measurement method 900 using a measurement apparatus disclosed herein (e.g., the measurement apparatus 100 in FIG. 1), according to embodiments of the present disclosure. The method 900 may be performed without moving a patient on the measurement apparatus during the measurement, and may include, but is not limited to, the following steps.

In step 904, a patient body is laid over a measuring aid imprinted on a fixed medium. As described above, the measuring aid includes a set of evenly spaced parallel lines that are divided at an intermediate parallel line into at least two sections (e.g., the sections 110-1 and 110-2 in FIG. 1) in a direction perpendicular to the intermediate parallel line, and the intermediate parallel line is a zero scale line of the set of evenly spaced parallel lines. The patient body may be laid over the measuring aid in the direction perpendicular to the intermediate parallel line.

In step 906, a top half of the patient body is measured by counting the number of parallel lines from the intermediate parallel line in a corresponding section of the at least two sections that is laid over by the top half of the patient body. For example, the top half may take up six parallel lines. Alternatively, the top half of the patient body may be measured by recording a number marked at the most distal parallel line from the intermediate parallel line in the corresponding section that is laid over by the top half of the patient body. For example, the most distal parallel line crossing the top of the patient's head may be marked 30, which indicates a distance or length from the intermediate parallel line to the most distal parallel line is 30 (inches or centimeters).

In step 908, the number of parallel lines laid over by the top half of the patient body is multiplied with a first corresponding measurement unit to obtain a length of the top half of the patient body. For example, if the first corresponding measurement unit is inch and the distance between each pair of adjacent parallel line is five inches, then in this example, the length of the top half of the patient body may be thirty inches (six lines multiplying five inches).

In step 910, a lower half of the patient body is measured by counting the number of parallel lines from the intermediate parallel line in another section of the at least two sections that is laid over by the lower half of the patient body. For example, the lower half may take up 5 parallel lines. Alternatively, the lower half of the patient body may be measured by recording a number marked at the most distal parallel line from the intermediate parallel line in the another section that is laid over by the lower half of the patient body. For example, the most distal parallel line crossing the bottom of the patient's feet may be marked 25, which indicates a distance or length from the intermediate parallel line to the most distal parallel line is 25 (inches or centimeters).

In step 912, the number of parallel lines laid over by the lower half of the patient body is multiplied with a second corresponding measurement unit to obtain a length of the lower half of the patient body. For example, if the second corresponding measurement unit is inch and the distance between each pair of adjacent parallel line is five inches, then in this example, the length of the lower half of the patient body may be twenty five inches (five lines multiplying five inches).

In step 914, the length of the lower half of the patient body and the length of the top half of the patient body are summed to obtain a total length of the patient body. In this example, the total length of the patient body is thirty inches plus twenty five inches to yield a length of fifty five inches. Alternatively, the total length of the patient body (55 inches) can be obtained by adding the number marked at the most distal parallel line crossing the top of the patient's head (30 inches) and the number marked at the most distal parallel line crossing the bottom of the patient's feet (25 inches).

As can be seen from the above, the parallel lines can be counted out from the zero line towards the head and towards the feet of the patient body. So that if the patient shifts up or down on the measurement apparatus, the measurement can be taken using the number at the foot and the number at the head, which can be added together to obtain the total distance (the total length) of the patient body. This procedure is unlike measuring conducted using an apparatus with a single set of ascending numbers (e.g., a traditional ruler or measuring tape) which requires positioning the patient's head or foot with the zero line or subtracting the offset from the zero line. By putting the zero line at or near the middle of the patient, the measurement can be calculated using addition which is less prone to error than subtraction. Therefore, the patient could be shifted one way or the other way relative to the zero line, without affecting the accurate measurement.

Accordingly, the present disclosure provides a measuring system for accurate, precise and uniform measurement of at least a portion of a patient body without moving the patient body. The system may comprise a fixed medium having a first surface and a second surface opposing the first surface, and a measuring aid imprinted on at least one of the first surface and the second surface. The measuring aid may include a set of evenly spaced parallel lines, a distance between each pair of adjacent parallel lines is configured to tabulate a total length of the portion of the patient body or a component thereof being measured, the set of evenly spaced parallel lines are divided at an intermediate parallel line into at least two sections in a direction perpendicular to the intermediate parallel line, the intermediate parallel line being a zero scale line of the set of evenly spaced parallel lines, each of the at least two sections is provided with individual scales of corresponding measurement unit for separately measuring discretely particular portions of the patient body, the set of evenly spaced parallel lines are parallel in a transverse direction of the fixed medium, and the intermediate parallel line is approximately located at a middle point of the fixed medium in a longitudinal direction of the fixed medium.

The present disclosure therefore may offer a nonobvious solution for standardized, clinical measurement of a patient body through the use of the measuring aid, which is divided into a particular number of sections or sets for precise measurement of individual portions of the patient body as well as the body as a whole in a variety of contexts. With a system and method for the use of the above-described measuring aid, true, accurate and consistent measurements of the patient body or portions thereof can be obtained for all manner of subjects, regardless of mobility, through the measurement of individual components of the body using the measuring aid, and, if necessary, the subsequent combination of or other calculation from discretely measured portions of the body to reach a final measurement for the total portion of the body desired to be measured.

Further, the measurement apparatus disclosed herein can be unique to each patient and does not suffer from the potential of being moved and contaminated by others. For example, the measurement apparatus disclosed herein need not be carried from one patient room to the next patient room, reducing contamination risk. Contamination risk and HIPAA compliance are a significant concern in a healthcare setting.

In addition, the measurement apparatus disclosed herein can make accurate and precise measurements of patients having movement restrictions. The risk of repositioning a patient for being appropriately oriented can be quite profound if the patient is, for example, intubated. For example, in a critical care setting (e.g., an intensive care unit) where a patient is intubated, paralyzed and/or sedated or if a patient has a spinal cord injury in a clinical setting, adjusting the patient to orient him/her can pose a quite significant risk to the patient. Also in a clinical or critical care setting where a patient may be overweight, moving the patient can be quite challenging, which not only exposes the patient to risks but also may place a healthcare worker in risk who is involved in moving the patient. Another example of patients placed in movement restrictions may be that a patient is on a mechanical ventilation. If the patient is to be moved, the breathing tube needs to be ensured to go with the patient's head without moving with respect to the patient head. If the breathing tube moves with respect to the patient's head, the patient could then potentially be placed in catastrophic situations which may actually be fatal. The same could be true if a patient has a large bore venous catheter in and is on continuous dialysis. In another example, a patient may be in a heart lung bypass, moving the patient could take out an arterial catheter which may cause the patient to bleed to death, or could take out a venous catheter which may also cause the patient to bleed to death. Therefore, moving a patient having movement restrictions or moving an unconscious critically ill patient may pose a significant risk to the patient. The measuring apparatus, system and method disclosed herein can alleviates that concern or risk, for example, by having a zero scale line in the middle of the fixed medium, such that an accurate and precise height or length measurement of a patient can be conveniently made by counting out from the zero scale line towards the head and the feet of the patient without requiring to move the patient body.

The present disclosure is not to be limited in terms of the particular embodiments described in this application, which are intended as illustrations of various aspects. Many modifications and variations can be made without departing from its spirit and scope, as may be apparent. Functionally equivalent methods and apparatuses within the scope of the disclosure, in addition to those enumerated herein, may be apparent from the foregoing representative descriptions. Such modifications and variations are intended to fall within the scope of the appended representative claims. The present disclosure is to be limited only by the terms of the appended representative claims, along with the full scope of equivalents to which such representative claims are entitled. It is also to be understood that the terminology used herein is for the purpose of describing particular embodiments only, and is not intended to be limiting.

The foregoing description, along with its associated embodiments, has been presented for purposes of illustration only. It is not exhaustive and does not limit the invention to the precise form disclosed. Those skilled in the art may appreciate from the foregoing description that modifications and variations are possible in light of the above teachings or may be acquired from practicing the disclosed embodiments. For example, the steps described need not be performed in the same sequence discussed or with the same degree of separation. Likewise various steps may be omitted, repeated, or combined, as necessary, to achieve the same or similar objectives. Accordingly, the invention is not limited to the above-described embodiments, but instead is defined by the appended claims in light of their full scope of equivalents.

In the preceding specification, various preferred embodiments have been described with references to the accompanying drawings. It may, however, be evident that various modifications and changes may be made thereto, and additional embodiments may be implemented, without departing from the broader scope of the invention as set forth in the claims that follow. The specification and drawings are accordingly to be regarded as an illustrative rather than restrictive sense.

What is claimed is:

1. A measuring apparatus for measurement of at least a portion of a patient body, comprising:
a measuring aid including a set of evenly spaced parallel lines, wherein a distance between each pair of adjacent parallel lines is configured to tabulate a length of the portion of the patient body or a component thereof being measured, the set of evenly spaced parallel lines are divided at an intermediate parallel line into at least two sections in a direction perpendicular to the intermediate parallel line, the intermediate parallel line is a zero scale line of the set of evenly spaced parallel lines, and each of the at least two sections is provided with individual scales of corresponding measurement units ascending from the intermediate parallel line for separately measuring particular portions of the patient body; and
a fixed medium having a first surface and a second surface opposing the first surface, wherein the measuring aid is imprinted on at least one of the first surface and the second surface, the set of evenly spaced parallel lines are parallel in a transverse direction of the fixed medium, and the intermediate parallel line is approximately located at a middle point of the fixed medium in a longitudinal direction of the fixed medium.

2. The measurement apparatus of claim 1, wherein
the corresponding measurement unit is at least one of a metric measurement system unit and an English measurement system unit.

3. The measurement apparatus of claim 1, wherein
the evenly spaced parallel lines in at least one of the at least two sections are further divided into a subset of smaller scale evenly spaced parallel lines.

4. The measurement apparatus of claim 1, wherein
the fixed medium is a bedsheet.

5. The measurement apparatus of claim 1, wherein
the fixed medium is a backboard for use in emergency medicine situations.

6. The measurement apparatus of claim 1, wherein
the measuring aid is imprinted on the first surface of the fixed medium; and
the second surface of the fixed medium is provided with at least one fastener.

7. The measurement apparatus of claim 6, wherein
the at least one fastener includes at least one of an adhesive patch and a hook-and-loop fastener.

8. The measurement apparatus of claim 6, wherein
the fixed medium is attached through the at least one fastener to a patient support apparatus, such that the fixed medium is secured to the patient support apparatus while the portion of the patient body is measured.

9. The measurement apparatus of claim 6, wherein
the fixed medium is attached through the at least one fastener to a backboard for use in emergency medicine situations, such that the fixed medium is secured to the backboard while the portion of the patient body is measured.

10. The measurement apparatus of claim 6, wherein
the fixed medium is attached through the at least one fastener to a stretcher for use in emergency medicine situations, such that the fixed medium is secured to the stretcher while the portion of the patient body is measured.

11. The measurement apparatus of claim 1, wherein
the measuring aid is imprinted on the first surface of the fixed medium;
the fixed medium is made of flexible materials; and
the fixed medium is adapted to spread over a medical examination table, the second surface of the fixed medium being in contact with a top surface of the medical examination table such that the portion of the patient body is measured using the measuring aid imprinted on the first surface of the fixed medium.

12. The measurement apparatus of claim 1, wherein
the set of evenly spaced parallel lines are configured to extend across the fixed medium in both the transverse direction and the longitudinal direction of the fixed medium, such that patient body extremities or medical conditions present on those extremities may be measured without moving the patient body.

13. The measurement apparatus of claim 1, wherein
the set of evenly spaced parallel lines are configured to extend across the fixed medium in both the transverse direction and the longitudinal direction of the fixed medium, such that medical devices being used to treat the patient body may be measured without moving the patient body.

14. A measuring system for accurate, precise and uniform measurement of at least a portion of a patient body without moving the patient body, comprising:
a fixed medium having a first surface and a second surface opposing the first surface, and a measuring aid imprinted on at least one of the first surface and the second surface, wherein
the measuring aid includes a set of evenly spaced parallel lines,
a distance between each pair of adjacent parallel lines is configured to tabulate a total length of the portion of the patient body or a component thereof being measured,
the set of evenly spaced parallel lines are divided at an intermediate parallel line into at least two sections in a direction perpendicular to the intermediate parallel line,
the intermediate parallel line is a zero scale line of the set of evenly spaced parallel lines,
each of the at least two sections is provided with individual scales of corresponding measurement unit for separately measuring discretely particular portions of the patient body,
the set of evenly spaced parallel lines are parallel in a transverse direction of the fixed medium, and
the at least two sections are configured to have approximately equal number of parallel lines on either side of the intermediate parallel line.

15. The measurement system of claim 14, wherein
the measuring aid is a first measuring aid; and
the measurement system further comprises a second measuring aid imprinted on the at least one of the first surface and the second surface on which the first measuring aid is imprinted,
wherein
the second measuring aid is configured to form an angle with respect to the first measuring aid,
the second measuring aid includes a second set of evenly spaced parallel lines,
a distance between each pair of adjacent parallel lines of the second set is configured to tabulate a total length of the portion of the patient body or a component thereof being measured,
the second set of evenly spaced parallel lines are divided at a second intermediate parallel line into at least two sections in a direction perpendicular to the second intermediate parallel line,
the second intermediate parallel line is a zero scale line of the second set of evenly spaced parallel lines,
each of the at least two sections of the second measuring aid is provided with individual scales of corresponding measurement unit for separately measuring discretely particular portions of the patient body,
the second set of evenly spaced parallel lines are parallel in a direction corresponding to the angle formed between the first measuring aid and the second measuring aid, and
the at least two sections of the second measuring aid are configured to have approximately equal number of parallel lines on either side of the second intermediate parallel line.

16. The measurement system of claim 14, wherein
the fixed medium is a bedsheet for use in patient care facilities.

17. The measurement system of claim 14, wherein
the fixed medium is a backboard for use in emergency medicine situations.

18. The measurement system of claim 14, wherein
the fixed medium is made of flexible materials; and
the measuring aid is repeatedly imprinted on the at least one of the first surface and the second surface of the fixed medium such that each measuring aid is approximately adapted to the dimension of a top surface of a medical examination table, wherein one of the first surface and the second surface is in contact with the top surface of the medical examination table such that the portion of the patient body is measured using a corresponding measuring aid imprinted on another of the first surface and the second surface of the fixed medium, thereby a section of the fixed medium used by a prior patient can be discarded and a new section of the fixed medium can be unrolled for a next patient.

19. A method of performing an accurate measurement of at least a portion of a patient body without moving the patient body, comprising:
having the patient body laid over a measuring aid imprinted on a fixed medium, wherein the fixed medium is configured to have a first surface and a second surface opposing the first surface, the measuring aid is imprinted on at least one of the first surface and the second surface, the measuring aid includes a set of evenly spaced parallel lines, a distance between each pair of adjacent parallel lines is configured to tabulate a total length of the portion of the patient body or a component thereof being measured, the set of evenly spaced parallel lines are divided at an intermediate parallel line into at least two sections in a direction perpendicular to the intermediate parallel line, the intermediate parallel line is a zero scale line of the set of evenly spaced parallel lines, each of the at least two sections is provided with individual scales of corresponding measurement unit for separately measuring discretely particular portions of the patient body, the set of evenly spaced parallel lines are parallel in a transverse direction of the fixed medium, and the intermediate parallel line is approximately located at a middle point of the fixed medium in a longitudinal direction of the fixed medium;
measuring a top half of the patient body by counting the number of parallel lines from the intermediate parallel line in a corresponding section of the at least two sections that is laid over by the top half of the patient body;
multiplying the number of parallel lines laid over by the top half of the patient body with a first corresponding measurement unit to obtain a length of the top half of the patient body;
measuring a lower half of the patient body by counting the number of parallel lines from the intermediate parallel line in another section of the at least two sections that is laid over by the lower half of the patient body;
multiplying the number of parallel lines laid over by the lower half of the patient body with a second corresponding measurement unit to obtain a length of the lower half of the patient body; and
summing the length of the lower half of the patient body and the length of the top half of the patient body to obtain a total length of the patient body.

20. The method of claim 19, wherein
the measuring aid is imprinted on the first surface of the fixed medium; and
the second surface of the fixed medium is provided with at least one fastener.

21. The method of claim 20, wherein
the at least one fastener includes at least one of an adhesive patch and a hook-and-loop fastener.

22. The method of claim 19, wherein
the fixed medium is at least one of a bedsheet for use in hospital units or hospital emergency departments, a backboard for use in emergency medicine situations, and a stretcher for use in emergency medicine situations.
